(19) United States
(12) Patent Application Publication
(10) Pub. No.: US 2021/0177731 A1
(43) Pub. Date: Jun. 17, 2021

(54) COSMETIC COMPOSITIONS IN PARTICULAR WITH ANTI-AGING EFFECT, COMPRISING A GREEN EXTRACT OF AFRAMOMUM ANGUSTIFOLIUM OR LONGOZA PLANT

(71) Applicant: LVMH RECHERCHE, Saint Jean de Braye (FR)

(72) Inventors: Carine NIZARD, Ivry-sur-Seine (FR); Olivier JEANNETON, Vitry-aux-Loges (FR); Emmanuelle LEBLANC-NOBLESSE, Donnery (FR); Virginie PECHER, La Chapelle Saint Mesmin (FR)

(21) Appl. No.: 16/764,754

(22) PCT Filed: Dec. 18, 2018
(86) PCT No.: PCT/FR2018/053367

§ 371 Date: May 15, 2020

(30) Foreign Application Priority Data
Dec. 22, 2017 (FR) 1762974

(51) Int. Cl. A61K 8/9783; A61Q 19/08; A61K 8/34

(52) U.S. Cl. CPC A61K 8/9783; A61K 2800/805; A61K 8/345; A61Q 19/08

(57) ABSTRACT

The present invention relates to a cosmetic composition.

This composition is characterized in that it comprises, as active ingredient, a green extract of seeds of the Aframomum angustifolium or longoza plant, obtained by extraction with a polar solvent of polyol, in particular a glycol, optionally in a cosmetically acceptable excipient.

This cosmetic composition has a good anti-aging effect.

The present invention essentially relates to a cosmetic composition, in particular with anti-aging effect, characterized in that it comprises, as active ingredient, a green extract of seeds of the Aframomum angustifolium or longoza plant.

The invention also relates to the use of a green extract of seeds of the Aframomum angustifolium or longoza plant, as a cosmetic agent, in particular for the manufacture of a cosmetic composition, which in particular has an anti-aging effect.

Hereinafter in the description and the claims, the term longoza will be used for simplification, but obviously it also means the Aframomum angustifolium plant.

Furthermore, green extract means an extract with a solvent providing ecological and cosmetic compatibility of the extract, for example a polar solvent comprising or consisting of a polyol, in particular a glycol, and more particularly chosen from propylene glycol, butylene glycol, pentylene glycol. This extract is also green, according to a particular embodiment, thanks to:

the use of a renewable plant resource consisting of longoza seeds;

the absence of waste solvents and in particular organic solvents such as hexane or heptane

the use of alternative green solvents used in cosmetics comprising or consisting of a polyol, in particular a glycol, and more particularly selected from propylene glycol, butylene glycol, pentylene glycol;

An extraction process that involves few steps with low energy consumption, in particular by performing a first cold extraction, a second extraction at a mild temperature of about 55° C.; and

The process should avoid bringing a color impact, keeping an industrial feasibility and without increasing the percentage of terpenes contained in the longoza, to avoid toxicological risks.

### RELATED ART

The article by E. M. Gaydou et al. published in Revue française des Corps Gras, No. 1 of January 1983, pages 21 to 25, a study on the fatty acid composition of oils extracted from seeds of some plants of Madagascar, including the Aframomum angustifolium plant (see Table 1, continuation 2, page 24). It is an extract of seeds of the plant by means of an apolar solvent, in particular using hexane (see page 24, “Experimental section”, first paragraph).

In addition, the patent ANDRE et al., LVMH RECHERCHE, U.S. Pat. No. 7,381,43662 describes a cosmetic composition containing an extract of seeds of the Aframomum angustifolium or longoza plant with a polar solvent consisting in practice of a hydroalcoholic ethanol/water mixture in a 70/30 volume ratio. This document describes an anti-aging effect by an action of stimulation of the synthesis of collagen, in particular collagen VII present at the dermal-epidermal junction or JDE, and/or by an action on the oxytalan fibers and/or the elaunin fibers; and also to protect the skin against aging resulting from the action of actinic radiation, in particular due to ultraviolet rays.

Also, the document US 2010/0055217 NIZARD, LVMH RECHERCHE is similar to the ANDRE patent above describing a hydroalcoholic extract of longoza but used with lipochronae-6 to obtain a synergistic effect.

In addition, the article by Wiley et al. in Cell Metab. 2016, 9 Feb.; 23(2):303-314, describes research results showing that mitochondrial dysfunction increases with age, which induces mitochondrial senescence with a different secretion phenotype.

### AIMS OF THE INVENTION

The invention aims at solving the technical problem of providing a solution for finding novel extracts or active ingredients from the Aframomum angustifolium or longoza plant, as cosmetic agents which have a good anti-aging effect or an improved anti-aging effect compared with previously known cosmetic anti-aging agents including the hydroalcoholic extract of the Aframomum angustifolium or longoza plant, and/or which have a very good compatibility in the context of topical application for cosmetic purposes, in particular to the skin, and/or which have no or substantially no side effects or irritant effects.

The invention also aims at solving the technical problem stated above with the help of novel extracts or active ingredients from the Aframomum angustifolium or longoza plant capable of slowing or preventing a mitochondrial dysfunction leading to mitochondrial senescence and premature aging of the skin.

Again, the invention aims at solving the above technical problems by also providing a process for obtaining the active ingredient which is environmentally friendly by avoiding the use of environmentally harmful solvents, which requires little energy, and which allows sufficient quantities to be prepared for use on an industrial scale and, in particular, on a cosmetic scale.

### SUMMARY OF THE INVENTION

The invention provides a solution to the technical problems set out above, in a simple, inexpensive manner which can be used on an industrial scale and, in particular, on a cosmetic scale.

According to a first aspect, the present invention provides a cosmetic composition, characterized in that it comprises, as active ingredient, an extract, called green extract, of seeds of the Aframomum angustifolium or longoza plant made with an ecologically friendly and cosmetically acceptable polar solvent, comprising or consisting of a polar solvent of a polyol, in particular a glycol, in a cosmetically acceptable excipient.

According to a second aspect, the invention also relates to the use of an extract, known as green extract, of seeds of the Aframomum angustifolium or longoza plant made with an environmentally friendly and cosmetically acceptable solvent, comprising or consisting of a polar solvent of a polyol, in particular a glycol, as a cosmetic agent, for the manufacture of a cosmetic composition.

According to a particular embodiment of the invention, the green extract of longoza is used as a cosmetic agent with anti-aging effect.

According to an advantageous alternative embodiment of the invention, the green extract of seeds of the longoza plant is used for an anti-wrinkle effect, in particular to diminish, eliminate, or slow the appearance of wrinkles, an action preserving or restoring the structure of the skin, in particular through an action of stimulation of the synthesis of ATP synthase 6 by the mitochondria.

Thus, the invention provides an effect of protection, repair and restoration of mitochondrial activity which prevents mitochondrial senescence resulting in an anti-aging effect, in particular anti-wrinkle.

The invention also protects the skin against aging resulting from the action of actinic radiation, in particular due to ultraviolet rays.

According to a third aspect, the invention relates to a novel process for extracting Aframomum angustifolium or longoza seeds.

In the context of the invention, the seeds of the Aframomum angustifolium or longoza plant are extracted with an ecologically friendly and cosmetically acceptable polar solvent, in particular a polar solvent comprising or consisting of a polyol, in particular a glycol, thus obtaining a “green” extract.

For each of the aspects of the invention, the polyol used may have the general chemical formula HO—R—OH, wherein R may represent a hydrocarbon radical —(CH₂)ₙ with n having from 2 to 16 carbon atoms, in particular from 3 to 8 carbon atoms.

According to a particular embodiment of the invention, the polyol used will be a common glycol, widely available in industry and inexpensive, such as propylene glycol, butylene glycol or 1,3-butylene glycol, pentylene glycol, and mixtures thereof.

In another alternative embodiment, the seeds are ground to a fine powder. The grains of this powder advantageously have a diameter of between 0.01 and 1 mm.

In yet another alternative embodiment, several successive extractions can be carried out until the material to be extracted is exhausted by the solvent polyol, in particular a glycol.

According to an alternative embodiment, extraction with the polyol, in particular a glycol, is carried out once at so-called “cold” room temperature and a second time at a temperature of about 55° C.

According to a particular embodiment of the invention, the extraction process comprises at least one step of extracting the ground dry seeds with a polar solvent consisting of 100% polyol, in particular a glycol. According to an alternative embodiment, the plant/solvent m/v ratio can be 1/1.75.

According to another alternative embodiment, before the extraction proper, the ground seeds are impregnated by soft maceration of for example 12 to 15 hours at room temperature in the polyol, in particular a glycol, thus increasing the accessibility and availability of the molecules of interest.

According to yet another alternative, after maceration, a first filtration is carried out to obtain a first filtrate and a cake of ground seeds, then the cake is extracted at a mild temperature, for example of about 55° C. for a period of several hours, for example about 3 hours, then filtration is carried out to obtain a second filtrate, for example on a filter with a porosity of 20-25 micrometers. According to a particular alternative, the two filtrates obtained are combined and can be filtered again on a sterilizing or non-sterilizing filter, for example from 0.5 to 0.1 micrometers. The solution obtained can be adjusted with the polyol, in particular a glycol, or other biocompatible and cosmetically acceptable solvent to give about 1% by weight of dry extract.

According to a particular embodiment of this extraction process, the extract in the polyol, in particular a glycol, may constitute in itself the active ingredient or agent according to the invention.

According to another particular alternative, it is possible to evaporate substantially completely the extraction solvent to obtain a dry extract which can either be used as such as the above-mentioned active ingredient or dissolved again in an environmentally friendly and cosmetically acceptable solvent, such as a polyol, in particular a glycol, or an alcohol or a hydroalcoholic mixture in order to obtain another alternative of the above-mentioned active ingredient.

According to another advantageous alternative of the invention, the solvent used for redissolution is a glycol.

According to a particular embodiment of the invention, as indicated above, a common glycol widely available in industry and inexpensive, such as propylene glycol, butylene glycol or 1,3-butylene glycol, pentylene glycol, and mixtures thereof, will be used. A particular alternative embodiment uses butylene glycol.

In the context of the invention, the extract obtained is easy to use as a cosmetic agent and to mix with the other ingredients of the cosmetic composition to be prepared, whether to be incorporated into an aqueous phase or a fat phase.

According to a fifth aspect, the present invention also relates to a cosmetic care process, characterized in that it comprises the topical application to the skin of a person in need thereof of a cosmetically effective amount of an extract, called green extract, of seeds of the Aframomum angustifolium or longoza plant, as previously defined.

According to an advantageous embodiment of the invention, this cosmetic care process is used to apply the seed extract of the above-mentioned Aframomum angustifolium or longoza plant to areas of the skin in need of anti-aging care, in particular to combat the effects of aging resulting from mitochondrial dysfunction.

It was unexpectedly discovered that the green extract of the glycol invention has a significant effect on energy metabolism. Adenosine triphosphate (ATP) synthase is an enzyme complex found in the mitochondrial cristae. The role of this enzyme is to synthesize ATP from the electrochemical gradient and adenosine diphosphate (ADP) which is maintained by the respiratory chain as well as inorganic phosphate (IP), to obtain ADP+IP=ATP.

ADP and IP are true molecular motors essential to the life of cells. ATP is the only form of chemical energy converted into chemical and mechanical energy.

The invention thus unexpectedly has an action preserving or restoring the structure of the skin, an effect of protection, repair and restoration of the elasticity and/or firmness of the skin, in particular by a stimulating action of the ATP synthase subunit 6.

The invention also has an anti-aging effect, which more particularly aims at an anti-wrinkle effect, to diminish, eliminate, or slow the appearance of wrinkles.

In any of the aspects of the invention, said green extract is present at a concentration, expressed as dry extract or substance, of between 0.001% and 5%, in particular between 0.01% and 1%, by weight based on the total weight of the composition.

Other aims, features and advantages of the invention will become clear to the person skilled in the art from the following description containing several embodiments of the invention provided by way of example and which in no way limit the scope of the invention.

In the examples below, all percentages are given by weight, temperature is room temperature, i.e. 25° C., or is given in degrees Celsius, pressure is atmospheric pressure, unless otherwise stated.

### EXAMPLE 1 ACCORDING TO THE INVENTION

Example of Laboratory Preparation of a Green Extract of Seeds of the Aframomum angustifolium or Longoza Plant, With a Glycol, for Example Butylene Glycol, According to the Invention,

From 200 g of longoza plant seeds from various commercial lots, the following extraction procedure is carried out:

a) first, the commercially available dried seeds are ground to an average powder size of, for example, 0.01 mm to 1 mm.

b) then, the powder is macerated overnight for about 12 hours in 350 mL of butylene glycol, or a seed/solvent ratio (w/v) of 1/1.75 at static room temperature.

c) after maceration, a first filtration is carried out, for example on a filter with a porosity of 20-25 micrometers, to obtain a first filtrate by obtaining an opaque orange-red solution with a dry residue around 20 g/L or maceration juice, and a cake of ground seeds, then

d) an extraction is carried out on the cake with a further 350 mL of butylene glycol, at a mild temperature, for example around 55° C. for a period of several hours, for example around 3 hours, and then

e) a second filtration is carried out, for example on a filter with a porosity of 20-25 micrometers, to obtain a second filtrate, obtaining an opaque orange solution with a dry residue of around 5 g/L.

f) According to a particular alternative, the two filtrates or extracts obtained are combined and may be re-filtered through a sterilizing filter, for example of 0.5 to 0.1 micrometers. The filtrate is a clear orangish-orange solution on which the active ingredient assay and thin-layer chromatography and microbiology can be carried out.

g) The filtrate or extract solution obtained may be adjusted with butylene glycol or other biocompatible and cosmetically acceptable solvent to give a dry matter content of 10 g/L or about 1% by weight of dry matter.

This butylene glycol extract constitutes in itself, according to a particular embodiment, the green extract of longoza or Aframomum angustifolium seeds according to the invention which can be used as a cosmetic agent or as an active ingredient for the manufacture of a cosmetic composition as shown in the following examples (1 g extract). This butylene glycol solution contains 1% by weight of dry extract.

h) for the incorporation of this dry extract into a cosmetic composition according to the invention, it is also possible to prepare a fresh solution thereof at the time of manufacturing said composition, in a cosmetically acceptable solvent, in particular in water, alcohol, 1,3-butylene glycol or a mixture of these solvents. This embodiment thus makes it possible to prepare solutions of dry green extract of the Aframomum angustifolium or longoza plant at the desired concentration.

i) finally, the various solutions of the extract described above may be packaged, pending their use in the manufacture of the desired cosmetic compositions. In this context, it is recommended to first carry out a sterilizing filtration on a filter with a pore diameter of 0.22 μm before packaging in bottles of appropriate volumes, then storage in a cold room, for example at 4° C.

On average, from 1 kg of ground longoza or Aframomum angustifolium seeds, about 3 to 3.5 kg of extract is obtained at 1% by weight of dry matter in butylene glycol, which corresponds to 30 to 35 grams of dry extract. The mass extraction yield obtained is on average 35% calculated as follows: mass yield =mass of dry extract contained in the final extract×100/mass of ground seeds introduced at the start.

### EXAMPLE 2 ACCORDING TO THE INVENTION

Example of Preparation on an Industrial Pilot Scale of a Green Extract of Seeds of the Aframomum angustifolium or Longoza Plant, With a Glycol, for Example Butylene Glycol, According to the Invention

### 1) Pilot-Scale Tests

The pilot-scale tests are designed to process 10 kg per pilot-scale test of Aframomum angustifolium seeds from Madagascar according to 3 lots of seeds identified G1, G2 and G3 for Pilot-scale tests 1, 2 and 3, and adequate filtration systems described below.

### 2) Extraction Procedure According to the Invention in Pilot-Scale Tests

The extract according to the invention is made with a polar solvent consisting of 100% butylene glycol from dry seeds of Aframomum angustifolium previously ground conventionally with a plant/solvent ratio (m/v) of 1/1.75 by first impregnating the plant by gentle maceration of 12 to 15 h at room temperature in butylene glycol to make the molecules of interest more accessible and available. A first filtration step is carried out to obtain a filtrate 1.

A second extraction is carried out at 55° C. on the cake for 3 h, then filtration is carried out on a filter with a porosity of 20-25 μm to obtain a filtrate 2.

The two filtrates 1 and 2 obtained are combined and filtered again on a 0.5 to 0.1 μm filter.

The resulting solution is adjusted with butylene glycol to be 1% dry extract.

The result is a clear orangish glycolic solution.

The 3 extracts of the 3 lots G1, G2 and G3, obtained with the 3 pilot-scale tests, are named E1, E2 and E3.

On average, 10 kg of ground seeds produces about 30 to 35 kg of extract at 1% DM in butylene glycol (which corresponds to 300 to 350 g of dry extract).

The mass extraction yield obtained is on average 35%. It is calculated as follows:

mass yield=mass of dry extract contained in the final extract×100/mass of ground seeds introduced at the start.

### 3) Physicochemical Analyses of Extracts

3-1) Comparable Dry Weight (DM): 0.96 to 1% (9.6 g/L to 10.0 g/L)

pH in the range of 6.6; Comparable color: 7.8 to 7.9 (Gardner)

Lab values 87.8-88.9; −3.3 to −2.5; 67.7 to 69.3.

3-2) Total polyphenols: European Pharmacopoeia method 2.8.14

The test dose is 0.05 g per sample. The reading is made at 750 nm in the visible.

Concentration (g/L) in
gallic acid equivalent

| | |
|---|---|
| E1 | 0.012 |
| E2 | 0.017 |
| E3 | 0.016 |

Contents between 0.012 and 0.016 g/L are obtained. It can be observed that the contents are slightly higher for the extracts used with the E2 and E3 seed extracts.

### 4) Determinations of Total Sugars

Anthrone-sulfuric acid assay method. The reading is taken at 620 nm.

Content (%) in
glucose equivalent

| | |
|---|---|
| E1 | 5.9 |
| E2 | 7.0 |
| E3 | 8.1 |

The contents are slightly higher for the extracts used with the E2 and E3 seed extracts.

### COMPARATIVE EXAMPLE 3 Between the Process According to the Invention of Example 2 Obtaining a Green Extract With Butylene Glycol From Seeds of the Aframomum angustifolium or Longoza Plant, According to the Invention and the Process According to Example 1 of U.S. Pat. No. 7,381,436 Obtaining an Alcohol/Water extract 70/30 by volume, from seeds of the Aframomum angustifolium or Longoza Plant

### 3-1) To Carry Out This Comparative Test, the Following 2 Samples Were Used:

a) a comparative lot of seeds of the Madagascar Aframomum angustifolium or longoza plant called lot SX000HH which is subjected to the extraction process described in Example 1 of U.S. Pat. No. 7,381,436 which uses as extraction solvent a hydroalcoholic ethanol/water mixture 70/30 by volume and the extract is called extract lot SX000HH.
b) the green extract E2 according to Example 2 of the above invention
   3-2) Determination of the Anti-Aging Effect of the Green Extract E2 of Example 2 of Longoza (Aframomum angustifolium) on Normal Human Keratinocyte (NHK) Cells by Action on the Synthesis of ATP Synthase Subunit 6 by Mitochondria

### 1. Introduction

A study on the transcriptional effects of butylene glycol green extract of longoza (Aframomum angustifolium) from Example 1 compared with the previous hydroalcoholic ethanol/water 70/30 volume extract according to Example 1 of the patent U.S. Pat. No. 7,381,436B2 was evaluated using the TaqMan low-density array (TLDA) technology. Using this technology, the modulation of the expression of 96 genes encoding specific proteins is studied in response to treatment of normal human keratinocytes.

After 24 hours of treatment, the cell monolayers were lysed to recover the RNAs. These RNAs were quantified and normalized to perform reverse transcription. The cDNAs obtained were then prepared for analysis on a NHF, NHK and NHM TLDA microfluidic card, 96 “chronobiology profile” genes.

### 2. Materials & Methods

### 2.1. Preparation of Tested Active Agents and Active Agent Controls

Active agent 1 according to the invention: 100% butylene glycol extract GREEN E2 from Madagascar longoza seeds according to Example 2 above.

Comparative active agent 2: extract with an EtOH/H₂O 70/30 v/v mixture of Madagascar longoza seeds according to Example 1 of U.S. Pat. No. 7,381,436B2 and Example 3-1 a) above.

The final concentrations tested are 0.0125% by weight for Active agent 1 and Active agent 2.

### 2.2. Cell Treatments

The study was performed on normal human keratinocytes from abdominoplasty from a 31-year-old female donor (code 1477). Cells were first seeded in complete Epilife medium (HKGS supplement) at passage 4 (P4), at a density of 125 000 cells per well, in 12-well plates. The cells were then treated with 0.0125% Perle de Madagascar Green and 0.0125% Perle de Madagascar as well as their respective excipient control (BG 100% and EtOH/H₂O 70/30 v/v).

After 4 hours of treatment, the cells are recovered in order to extract the total RNA.

### 2.3. TaqMan Low-Density Array (TLDA) Technique

### 2.3.1. Total RNA Extraction Using Microlab STAR (Hamilton)

The cell culture medium is removed, and 250 μL of RLT lysis buffer (provided in the Nucleospin RNA trace kit, (product code 740709, lot number 1412/001, Macherey-Nagel) is added. The cells are scraped with a cell scraper and then the cell lysate, recovered in a 1.2 mL deep-well (provided in the Nucleospin RNA kit). Total RNA is extracted according to previously developed protocols (NS_96RNA_KHN or NS_96RNA_FHN.dws). The total RNA solutions obtained are assayed, and their quality checked, using a microplate reader, the SPECTROstar Nano (BMG Labtech) coupled to the Microlab STAR. This device is connected to the computer controlling the robotic platform and has specific software for analyzing the results (MARS software). The technique requires a 384-well microplate (384 micro-clear plate black LoBase, product code: 788876 lot: 1305055 Greiner), a positive control (RNA 250, product code: AM7155, lot: 1301028 (NHF), lot: 1208025 (NHK), lot: 1208026 (NHM), Thermofisher) to validate the pipetting performed by the robot as well as the values generated by the SPECTROstar Nano reader.

### 2.3.2. Synthesis of Complementary DNA

The reverse transcription (RT) kit used is the High-Capacity Reverse Transcription Kit (product code: 4368813, lot: 00288647; Thermo Fisher) and was used according to the protocol provided: 500 ng total RNA is diluted in water to a final volume of 25 μL. It is then incubated for 10 minutes at 25° C. then 2 hours at 37° C. in the presence of 25 μL of High-Capacity Reverse Transcription Kit 2× reaction mixture previously prepared as shown below. The different incubations are carried out in the TRobot (Biométra).

TABLE 2
Preparation of the High-Capacity Reverse Transcription Kit 2X
reaction mixture for 1 reaction

| | | | Random | RNase | | |
|---|---|---|---|---|---|---|
| | RT | | primer | out | | |
| Reagents | buffer | dNTP | probe | enzytne | RT | H₂O |
| Volume | 5 μL | 2 μL | 5 μL | 0.5 μL | 2.5 μL | 10 μL |

### 2.3.3. PCR-TaqMan Low-Density Array

15 μL of each RT is mixed with 60 μL water then 75 μL TaqMan Gene Expression Master Mix (product code: 4369016, lot: 1507235; Thermo Fisher) is added. After homogenization, 100 μL is deposited on the 96-gene chronobiology profile microfluidic cards (lot B1885 (NHF), lot B1884 (NHK) and lot B1942 (NHM)), which are centrifuged and then sealed.

The CD, corresponding to the profile of the genes deposited on the plates, is loaded into the SDS 2.4 software, which thus makes it possible to specify the location of each gene on the card. The control genes (called “endogenous” genes in the software) to be used for normalization of results are indicated before the PCR is started. PCR is performed according to the protocol provided by Applied Biosystems in the ABI Prism 7900HT Sequence Detection System. The qPCR steps are 2 min at 50° C., 10 min at 94.5° C. then 30 min at 97° C. and 1 min at 59.7° C. for 40 cycles.

### 2.3.4. Statistical Analysis of Results

Real-time quantitative PCR can be exploited if its efficiency is between 90% and 110%.

In the RT-PCR/TLDA method, quantitation is performed using the comparative method of ΔCt, i.e., relative quantitation (RQ). This method determines the Ct of each gene on the card using the RQ Manager software which takes into account the background noise for each gene. This Ct is normalized to the Ct of a housekeeping gene (in our study beta-2-microglobulin) which is studied in parallel on the card. This gene being invariant in the cells, its variations reflect the variability of the experiment (total RNA assay, pipetting, reverse transcription step, deposits in the microfluidic card, quality of the card, PCR in the apparatus). With this RQ Manager software, we obtain the RQ (Relative Quantitation) values which correspond to the amplitude level (× times greater or smaller than the control) of the expression compared with our control. The latter can be the untreated control or the excipient. The RQ is obtained by the following calculation (the value of the untreated being equal to 1):

RQ=2−ΔΔCt=2−(ΔCt treated−ΔCt untreated)

ΔCt treated=Ct treated target gene−Ct treated housekeeping gene

ΔCt untreated=Ct untreated target gene−Ct untreated housekeeping gene

For this method to be reliable, it is imperative that the amplification efficiency of the gene of interest and the housekeeping gene be the same. This was verified during the development process prior to routine use of the plates.

Student's t-test will be used to evaluate statistically significant variations in transcriptional activity. Each condition is performed in triplicate (3 untreated and 3 treated under the same conditions). Fisher's F-test is first applied by comparing the two data matrices. When the value is greater than α=0.05 then the variance of Student's t-test is 2, when Fischer's F-test is less than α=0.05 then the variance will be equal to 3. The transcriptional variations selected will be those corresponding to a Student's t-test lower than α=0.05.

A criterion taking into account the expected effect (+ for an increase, − for an inhibition) allows a first interpretation of results to be made
if the effect obtained goes in the expected direction then the grayscale code is applied as above
if the result obtained is the opposite of the expected effect, the gray scale code is not applied but the result remains visible since it is a significant result.

All these data allow us to note the extracts in relation to each other since the Excel macro indicates the number of genes modulated in the expected direction/number of modulated genes in total. In the context of a screening, this score can be used as a selection criterion.

Results Relating to the Effect of Longoza Green butylene Glycol Extract E2 According to the Invention of Example 2, Compared with Longoza Prior to the Ethanol/Water 70/30 Vol/Vol Hydroalcoholic Mixture of U.S. Pat. No. 7,381,43662 Example 1, on Normal Human Keratinocytes (NHK) After 4 h.

The longoza green E2 according to Example 2 of the invention increases in an unexpected manner by a coefficient of +2.697 the synthesis of ATP synthase subunit 6 by NHK mitochondria.

In conclusion of the above, thanks to the above test on NHK, it was possible to demonstrate the significantly repairing effect of the mitochondrial dysfunction of the green butylene glycol extract of longoza according to the invention, which combats the senescence of mitochondrial cells and therefore combats skin aging.

Thus, the invention can be used as a cosmetic agent for the preparation of a cosmetic composition for topical use to preserve the mitochondria and to combat skin aging.

Various examples of formulations of cosmetic compositions are given below by way of illustration and therefore in no way limit the scope of the invention.

### EXAMPLE 4 ACCORDING TO THE INVENTION

Cosmetic gel for improving the firmness of the face

| | |
|---|---|
| butylene glycol 100% green extract of longoza | 2% |
| obtained in Example 1, g) | |
| glycol | 3% |
| commercially available AMPS polymer, | 3% |
| (trade name Sepigel 305 ®) | |
| hydrogenated castor oil (Cremophor CO-60 ®) | 2% |
| polyethylene glycol | 1.5% |
| preservative | 0.5% |
| perfume concentrate | 0.3% |
| UV filter (benzophenone-4) | 1% |
| water, q.s. | 100% |

This gel can be applied to the face once a day for several weeks in order to improve the firmness of the facial skin, especially on areas with wrinkles, thus observing at the end of this treatment, a restoration of suppleness with a clear effect of skin rejuvenation and attenuation or disappearance of these wrinkles.

### EXAMPLE 5 ACCORDING TO THE INVENTION

Anti-wrinkle day cream in the form of an emulsion

| | |
|---|---|
| butylene glycol 100% green extract E1 of longoza | 2% |
| obtained in Example 2 | |
| steareth-21 (Brij 721) | 2.5% |
| glyceryl stearate (Tegrin) | 1.1% |
| stearyl alcohol | 5% |
| glycerol tricaprylate/caprylate | 12.5% |
| butylene glycol | 3% |
| glycerin | 2% |
| preservative | 0.5% |
| perfume concentrate | 0.5% |
| UV filter (octyl methoxycinnamate) | 7.5% |
| water, q.s. | 100% |

### EXAMPLE 6 ACCORDING TO THE INVENTION

Anti-wrinkle tonic lotion

| | |
|---|---|
| butylene glycol 100% green extract E2 of longoza | 2% |
| obtained in Example 2 | |
| butylene glycol | 3% |
| EDTA | 0.1% |
| solubilizer | 1% |
| perfume concentrate | 0.3% |
| ethanol | 5% |
| UV filter (benzophenone-4) | 0.13% |
| water, q.s. | 100% |

### EXAMPLE 7 ACCORDING TO THE INVENTION

Ultraviolet protective make-up powder for the face

| | |
|---|---|
| butylene glycol 100% green extract E3 of longoza | 0.25% |
| obtained in Example 2 | |
| talcum | 17% |
| mica | 20% |
| sericite | 20% |
| pigments | 8% |
| organic powder (nylon) | 20% |
| silica | 8.75% |
| mineral oil or silicone | 3% |
| UV filter (octyl methoxycinnamate) | 3% |

### EXAMPLE 8 ACCORDING TO THE INVENTION

Anti-wrinkle treatment foundation

| | |
|---|---|
| butylene glycol 100% green extract E2 of longoza | 2% |
| obtained in Example 2) | |
| 4-polyglyceryl isostearate and cetyl dimethicone | 5.1% |
| copolyol and hexyl laurate | |
| cyclopentasiloxane and cyclohexasiloxane | 5.0% |
| cetyl dimethicone | 1.0% |
| caprylic/capric triglycerides | 2.2% |
| octyl stearate | 1.4% |
| mineral oil | 3.5% |
| hydrogenated castor oil | 1.2% |
| beeswax | 0.8% |
| polymethyl methacrylate | 1.1% |
| iron oxides | 0.45% |
| titanium dioxides | 5.2% |
| NaCl | 0.6% |
| perfume concentrate | 0.1% |
| UV filter (octyl cinnamate) | 3% |
| water, q.s. | 100% |

1-10. (canceled)

11. A cosmetic composition comprising, as one active ingredient, an extract, called green extract, of seeds of the Aframomum angustifolium or longoza plant made with an ecologically friendly and cosmetically acceptable polar solvent, comprising, or consisting essentially of, a polar solvent of polyol, in a cosmetically acceptable excipient.

12. The composition as claimed in claim 11, wherein the polyl comprises a glycol.

13. The composition as claimed in claim 11, wherein the extract from the seeds of the plant is obtained by an extraction process comprising at least one step of extracting the ground seeds with the polar solvent of polyol.

14. The composition as claimed in claim 11, wherein the extract from the seeds of the plant is dissolved again in an environmentally friendly and cosmetically acceptable solvent comprising a polyol, or an alcohol or a hydroalcoholic mixture.

15. The composition as claimed in claim 11, wherein the polyol has the general chemical formula HO—R—OH, wherein R represents a hydrocarbon radical —(CH₂)ₙ with n having 2 to 16 carbon atoms.

16. The composition as claimed in claim 11, wherein the polyol has the general chemical formula HO—R—OH, wherein R represents a hydrocarbon radical —(CH₂)ₙ with n having 3 to 8 carbon atoms.

17. The composition as claimed in claim 11, wherein the polyol comprises a glycol selected from propylene glycol, butylene glycol or 1,3-butylene glycol, pentylene glycol, and mixtures thereof.

18. The composition as claimed in claim 11, wherein said extract of longoza seeds is present at a concentration, expressed as dry extract, of between 0.001% and 5%, by weight based on the total weight of the composition.

19. The composition as claimed in claim 11, wherein said extract of longoza seeds is present at a concentration, expressed as dry extract, of between 0.01% and 1%, by weight based on the total weight of the composition.

20. A cosmetic care process, comprising at least one topical application to the skin of a person in need thereof of a cosmetically effective amount of an extract, called green extract, of seeds of the Aframomum angustifolium or longoza plant made with an ecologically friendly and cosmetically acceptable polar solvent, comprising, or consisting essentially of, a polar solvent of polyol, in a cosmetically acceptable excipient.

21. The process as claimed in claim 20, wherein the at least one topical application of the seed extract of Aframomum angustifolium or longoza plant is carried out on the areas of the skin in need of a cosmetic care selected from the group consisting of an anti-aging care, of an anti-wrinkle care to reduce, eliminate or slow down the appearance of wrinkles, of an anti-aging care to combat the effects of aging resulting from mitochondrial dysfunction, of an action preserving or restoring the structure of the skin, of a protective, repairing or restoring effect on the elasticity or firmness of the skin, and of a stimulating action of the ATP synthase subunit 6.

22. The process as claimed in claim 20, wherein the polyl comprises, or is consisting essentially of a glycol.

23. The process as claimed in claim 20, wherein the extract from the seeds of the plant is obtained by an extraction process comprising at least one step of extracting the ground seeds with the polar solvent of polyol.

24. The process as claimed in claim 20, wherein the extract from the seeds of the plant is dissolved again in an environmentally friendly and cosmetically acceptable solvent comprising, or consisting essentially of a polyol, or an alcohol or a hydroalcoholic mixture.

25. The process as claimed in claim 20, wherein the polyol has the general chemical formula HO—R—OH, wherein R represents a hydrocarbon radical —(CH₂)ₙ with n having 2 to 16 carbon atoms.

26. The process as claimed in claim 20, wherein the polyol has the general chemical formula HO—R—OH, wherein R represents a hydrocarbon radical —(CH₂)ₙ with n having 3 to 8 carbon atoms.

27. The process as claimed in claim 20, wherein the polyol comprises a glycol selected from propylene glycol, butylene glycol or 1,3-butylene glycol, pentylene glycol, and mixtures thereof.

28. The process as claimed in claim 20, wherein said extract of longoza seeds is present at a concentration, expressed as dry extract, of between 0.001% and 5%, by weight based on the total weight of the composition.

29. The process as claimed in claim 20, wherein said extract of longoza seeds is present at a concentration, expressed as dry extract, of between 0.01% and 1%, by weight based on the total weight of the composition.

30. The composition as claimed in claim 12, wherein the polyol consists essentially of the glycol.
